(19) United States
(12) Patent Application Publication
(10) Pub. No.: US 2011/0071603 A1
(43) Pub. Date: Mar. 24, 2011

(54) Cyclic Heat Therapy Device

(76) Inventor: Joshua John Edward Moore, Elora (CA)

(21) Appl. No.: 12/888,406

(22) Filed: Sep. 22, 2010

Related U.S. Application Data
(60) Provisional application No. 61/245,651, filed on Sep. 24, 2009.

(51) Int. Cl. A61F 7/00

(52) U.S. Cl. 607/96

(57) ABSTRACT

The therapy device enables the automatic application of customized and well controlled contrast therapy to localized areas of human and animal bodies. An electronic control circuit is used in conjunction with heat dissipating and heat absorbing surfaces to control the operation of a thermoelectric cooling module (TEC) to enable the administering of localized contrast therapy on human and animal bodies. The use of temperature sensors, timing circuits, and microprocessors in connection with the TEC and heat absorbing and dissipating surfaces allows precise control of the applied temperature and the time duration for cooling and heating cycles. The device is programmable and is capable of automatic cycling between desired heating and cooling temperatures for variable lengths of time.

### CROSS-REFERENCE TO RELATED APPLICATIONS

This application claims the benefit of U.S. Provisional Patent Application Ser. No. 61/245,651 filed Sept. 24, 2009 and entitled CYCLIC HEAT THERAPY DEVICE, the contents of which are herein incorporated by reference in their entirety.

### BACKGROUND OF THE INVENTION

Many injuries to the human body such as sprains, damaged ligaments or tendons, torn or bruised muscles, aching joints, and post surgery recoveries are commonly treated with the application of hot and or cold therapy. Such therapy involves the application of heat or removal of heat to the afflicted area of the human body. This temperature treatment helps to reduce swelling, pain, and promote healing. In many instances it is also recommended that cyclic heat treatment, hot-cold-hot-cold, or cold-hot-cold-hot-cold-hot or similar patterns offers superior health benefits over that of constant heat or constant cold treatments. In the past heat treatments have been applied using hot water bottles, ice bags, frozen gels, microwave materials, and disposable chemical bags to produce exothermic or endothermic reactions. Large hot and cold baths have been used to provide contrast therapy for athletes but these systems are large, expensive, and there are patient compliance issues associated with moving persons from hot to cold baths and with non-localized treatment of the affected area of the body. Smaller systems are available that use heated or cooled water and circulate the fluids through tubes around the afflicted areas of the body to provide cyclic relief, but these are bulky, expensive, and have slow cycling times due to large volume fluid changeover. All of these cyclic contrast therapy systems have disadvantages.

One common limitation to heat treatment using the present devices is the need for prepared devices. For example, ice bags and frozen gels need to be frozen and stored in a freezer prior to the occurrence of an injury to be useful as a cooling aid. This requires freezer space and precognitive thought that injury may occur. Likewise, a hot water bottle and some heat gels need to be placed in boiling water to reach an effective hot temperature. The use of cold packs and ice bags also results in significant condensation and moisture build up. Since these types of devices cool in all directions moisture from the air can drip on floors or saturate clothing and fabrics. Chemical hot and cold packs solve the issue of preparing the devices in advance but are only one time use. By breaking chemical packets inside a bag a chemical reaction takes place to produce the desired hot or cold effect. However, this chemical solution is designed for either hot or cold and specific bags are needed for each application. The chemical reaction is not reversible and its operating life is limited to the length of the chemical reaction. Once the reaction is completed the bags are useless and must be discarded. There are some heat blankets and wraps available that can provide hot therapy, but there is no portable device available that can produce both hot and cold therapy in cyclic succession. With the above mentioned examples there is no way to cycle between hot and cold quickly without manually alternating between heating and cooling devices. Similarly, there is poor control over the temperatures and durations that the devices operate. A need exists, therefore, for a device that can automatically cycle between hot and cold treatments providing heating, cooling, and contrast therapy relief with accurate temperature and cycling duration controls.

### BRIEF DESCRIPTION OF THE DRAWINGS

To further clarify certain aspects of the present invention, a more detailed description of the invention will be rendered by reference to example embodiments thereof which are disclosed in the appended drawings. It is appreciated that these drawings depict only example embodiments of the invention and are therefore not to be considered limiting of its scope. Aspects of the invention will be described and explained with additional specificity and detail through the use of the accompanying drawings in which:

FIGS. 1A-1D disclose various views of an example cyclic heat therapy device shown with an example wrap. More specifically FIG. 1D shows an exploded view of an example cyclic heat therapy device;

FIGS. 2A-2B disclose various views of an example cyclic heat therapy device from FIG. 1 showing the cyclic heat therapy device with the wrap removed. More specifically FIG. 2B shows an exploded view of an example cyclic heat therapy device; and

FIG. 3A discloses a view of an example cyclic heat therapy device from FIGS. 1 and 2 showing the device in application on a human hand.

### DETAILED DESCRIPTION OF EXAMPLE EMBODIMENTS

Example embodiments of the present invention relate to an example cyclic heat therapy device used to heat or cool human, animal, or similar bodies by external application of the device. The example cyclic heat therapy device disclosed herein can aid in the localized temperature control of joints, muscles, tendons, ligaments, or any part of a human or animal body that is contacted by the device. The example cyclic heat therapy device is capable of both cooling and heating in succession on demand and repeating in a cyclic manner with precise temperature and cycle duration control. The device is capable of being used in a heating only mode, a cooling only mode, or a cyclic mode where the output surface temperature of the device cycles between hot and cold phases. The novel stacked configuration of this invention having the control circuits between the heat dissipating and the heat absorbing surfaces allows use without the need for additional cooling fans or fluids. This reduces power consumption and overall package size and allows for internal temperature monitoring of both the heat dissipating and heat absorbing surfaces by the control circuits.

Reference will now be made to the drawings to describe various aspects of exemplary embodiments of the invention. It is to be understood that the drawings are diagrammatic and schematic representations of such exemplary embodiments, and are not limiting of the present invention, nor are they necessarily drawn to scale.

### 1. Example Cyclic Heat Therapy Device

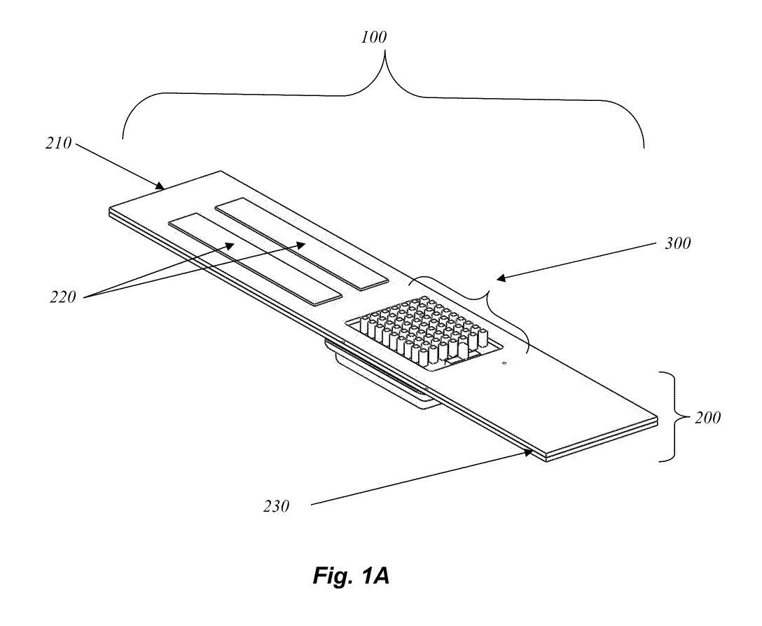

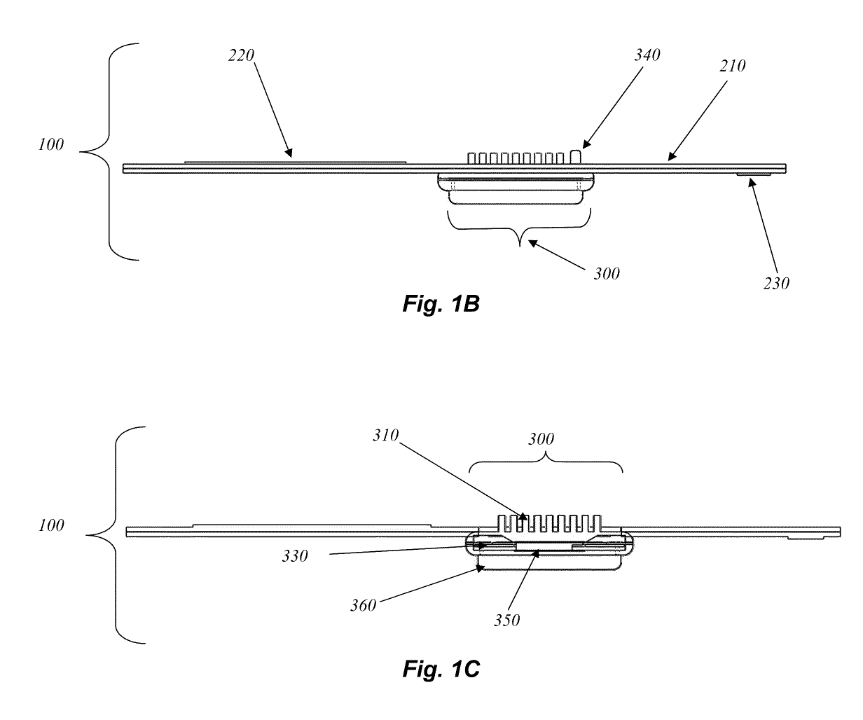

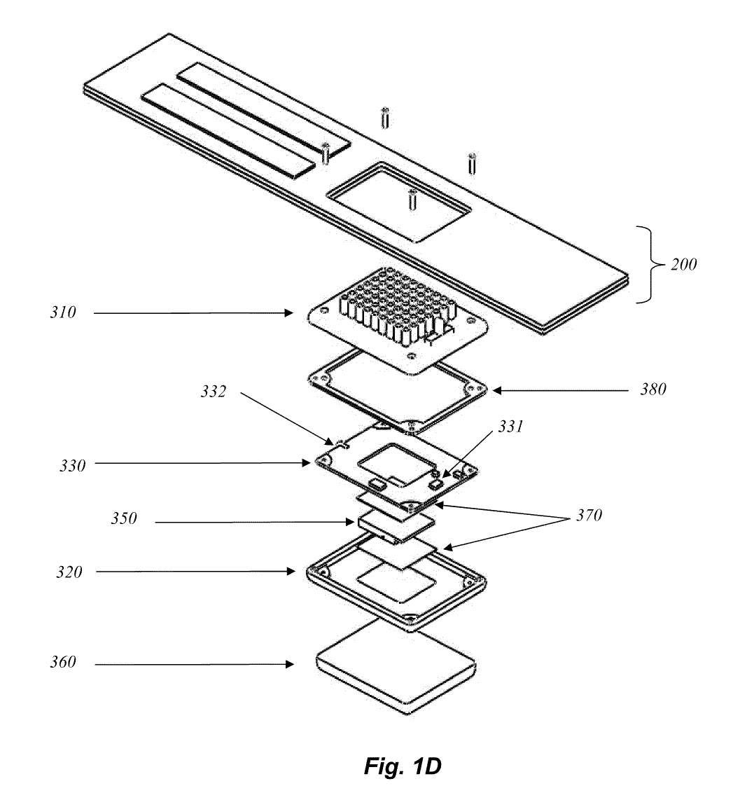

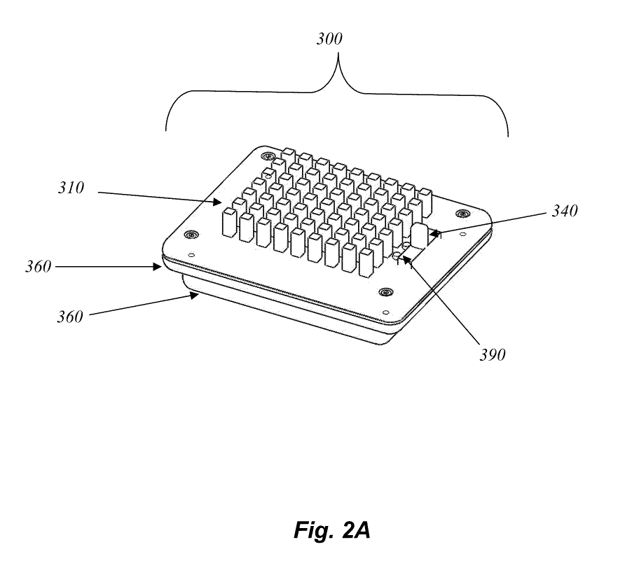

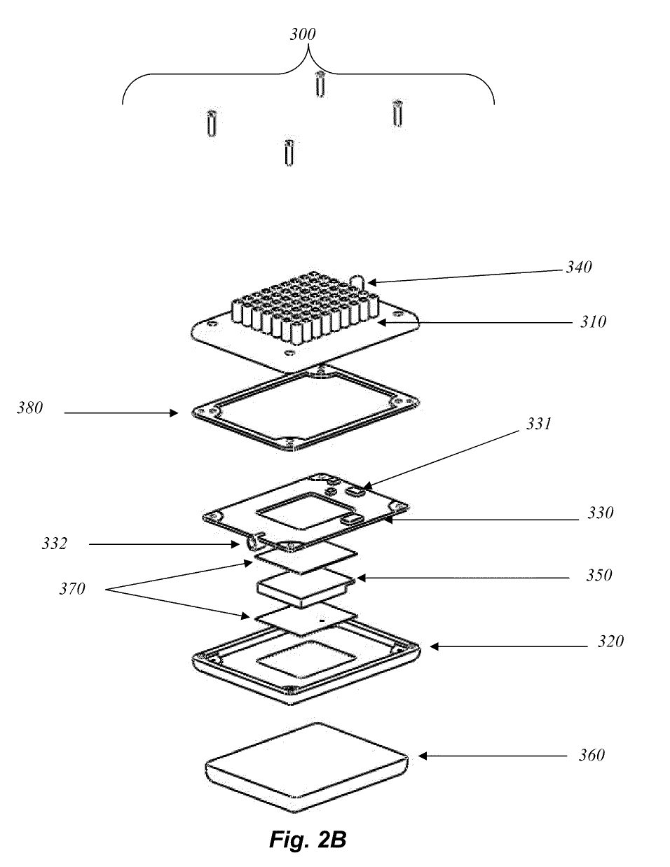

With reference first to FIG. 1, aspects of an example cyclic heat therapy device are disclosed. In particular, an example embodiment of a cyclic heat therapy device is shown. FIGS. 1A through 1C shows multiple views of the cyclic heat therapy device 100. The heating and cooling unit 300 is secured to the human body (not shown) using a flexible wrap 200. Shown is an example wrap made from neoprene or similar flexible rubber covered with a fabric 210 and secured to the human body (not shown) using hook 220 and loop 230 or other fastener materials which may include snaps, buckles, tied fabric, laces, or other fastener materials. The wrap 200 shown is a generic wrap. In another embodiment of the wrap 200 the wrap is designed to secure the heating and cooling unit 300 to a shoulder, back, or other body part that requires a unique wrap configuration and the wrap 200 would then take on a shape corresponding to that body part. The heating and cooling unit 300 can also be considered to be representative of the present invention without a wrap 200. In such an embodiment as shown in FIGS. 2A and 2B, and explained in more detail in the following section, the heating and cooling unit 300 is secured to the body by the human or external interface surface 360 using adhesive pads, gels, pastes, tapes or similar tacky medium which would provide the thermal contact and holding strength for the unit to the body. In such a case no additional wrap would be required. FIG. 1D shows an exploded view of the example cyclic heat therapy device 100. In particular the view shows the various components included in the heating and cooling unit 300 which will be explained in more detail in FIGS. 2A-2B. The cyclic heat therapy device is not limited to the example configurations disclosed in FIGS. 1A-1D. In another embodiment the cyclic heat therapy device could consist of one or more heating and cooling units 300 in various wrap 200 configurations or without wraps and adhered to the body using multiple external interface surfaces 360.

### 2. Example Cyclic Heat Therapy Device

With reference to FIGS. 2A and 2B, aspects of an example cyclic heat therapy device heating and cooling unit 300 are disclosed. In particular, the example cyclic heat therapy device heating and cooling unit 300 consists of a heat sink 310, a thermoelectric module also known as a thermoelectric cooler (TEC) 350, a printed circuit board (PCB) 330, a thermally conductive base 320, a thermally conductive compliant external interface surface 360, thermally conductive pads 370, a switch 340, and indicator lights 390. FIG. 2A shows an assembled heating and cooling unit 300 without a wrap 200. From this view the top surface shows the heat sink 310, the conductive base 320 and the external interface surface 360. Both the heat sink 310 and the conductive base 320 are made from machined aluminum, but could also be made from copper or any other material with a high thermal conductivity. The heat sink 310 and the conductive base 320 could also be produced using casting, injection molding, stamping or other suitable processes. As shown the human interface material 360 is made from a compliant thermally conductive material such as a thermal gel in a pouch or other flexible thermally conductive material. The external interface surface 360 could also be produced from rigid thermally conductive materials such as aluminum or copper or be integrated as part of the conductive base 320. In one embodiment the external interface surface 360 has adhesive properties and is used to hold the heating and cooling unit on the body. As stated in section 1 this could include pads, gels, pastes, tapes or other similar tacky medium which would provide the thermal contact and holding strength for the unit to the body. Also shown in FIG. 2A is a user switch 340 that allows the user to select the operating mode of the heating and cooling unit 300. Example heating and cooling modes include: 1) always heat, 2) always cool, 3) cycle between hot and cold or between cold and hot, and 4) off. It can also be envisioned that additional operating modes or functionality could be added to the device such as user controlled options like temperature and cycle time settings. Additional predefined modes could also be included such as certain cycling time and temperature combinations could be encoded into the device and would then be selectable by additional positions on a switch 340 or by actuation of additional switches. In this embodiment only one user input device is shown represented by the user switch 340. It can be envisioned that one or more switches, dials, buttons, or touch screens could be included within the scope of this device for use as user input or operational displays. Indicator lights 390 are also shown which produce different colors depending on the different operating modes. For example blue light indicates cooling, red light heating, and flashing red or flashing blue for cycling mode heating or cooling. It can be envisioned that visual display screens could be used as replacement for or in addition to the indicator lights 390 to provide similar user feedback about operating conditions, cycle times, temperatures, or modes of operation. These operating modes and lighting configurations given are for example only are not limiting of the scope of the cyclic heat therapy device 100.

FIG. 2B is an exploded view of the construction of an example heating and cooling unit 300 for an example cyclic heat therapy device. The construction shows a stacked configuration where the TEC 350 is sandwiched using thermal pads 370 between the heat sink 310 and the external interface surface 360. The conductive base 320 and the heat sink 310 protects and contains the PCB 330 which is used to control the operating mode, mode duration, and temperatures of the cyclic heat therapy device. The PCB 330 includes circuitry and electrical components 331 such as a 555 timer, microprocessor and other timing and switching circuits that enables the TEC 350 to switch automatically between heating and cooling operations when the cyclic heat therapy device is in the cycle operating mode. As part of the circuitry and electrical components 331 the PCB also contains temperature sensing components on the primary and secondary sides which are in direct thermal contact with the heat sink 310 and the conductive base 320. This enables the circuitry to monitor and reliably control the temperatures of the heat sink 310 and the conductive base 320 at the desired temperatures. As a safety feature the circuitry can then disable the device if the temperatures exceed a programmed high or low value. The timing and temperature sensing circuitry allows the cycle times and temperatures for the device to be set and reliable controlled. Such control allows different contrast therapy programs to be programmed for different treatments or injury types.

In the heating mode the portion of the TEC 350 contacting the conductive base 320, known here as the bottom portion, becomes hot and warms the external interface surface 360. In the cooling mode the portion of the TEC 350 that is contacting the conductive base 320, the bottom portion, becomes cold and cools the external interface surface 360. The heat that is removed from the human body contacting the external interface surface 360 is transferred through the TEC 350 to the portion of the TEC 350 contacting the heat sink 310, known here as the top portion, and dissipated to the ambient air by the heat sink 310. The PCB 330 also includes a DC power jack 332 that is used to supply electrical power to the heating and cooling unit 300 from an AC/DC adapter (not shown).

The example configuration of a heating and cooling unit 300 for a cyclic heat therapy device is not limiting of the example shown. It is also envisioned that this device can run on a battery supply. In this alternate embodiment of a cyclic heat therapy device the battery could be included within the heating and cooling unit 300, attached to the wrap 200 or otherwise externally mounted and connected through the DC power jack 332 on the PCB 330. The use of a battery pack would allow increased portably for the cyclic heat therapy device.

### 3. Example Cyclic Heat Therapy Device

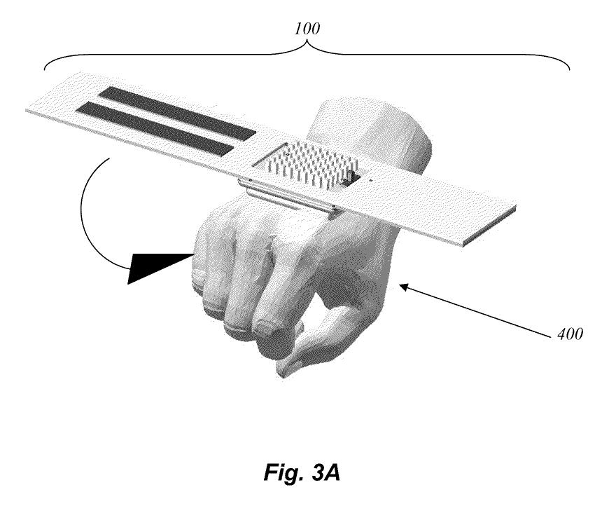

With reference now to FIG. 3A, an example cyclic heat therapy device in application is disclosed. In general, the example cyclic heat therapy device 100 is shown being installed onto a human hand 400. In this embodiment the wrap 200 will be secured to the hand 400 by putting the two ends of the wrap together and securing the hook 220 and loop 230 fasteners together. The external interface surface 360 makes contact to the back of the hand and the heat sink 310 faces out towards the environment. This enables the user to select a heating, cooling, or cyclic heating and cooling setting for the example cyclic heat therapy device. In an alternate embodiment the adhesion surface of the external interface surface 360 provides the holding strength for the heating and cooling unit 300 to the hand 400 and the wrap is not needed. The external interface surface 360 then transfers heat to, or removes heat from, the affected area of the body depending if the unit is operating in the heating or cooling portion of the contrast therapy cycle. The application shown in FIG. 3A is not limiting of the scope of the cyclic heat therapy device. For example, shoulders, back, neck, forehead, ankles knees or other parts of human or animal bodies could all be treated by the device. The device can be used on any part of the body where selective heating, cooling, or cyclic heating and cooling are desired.

The example cyclic heat therapy device may be embodied in other specific forms without departing from its spirit or essential characteristics. The described embodiments are to be considered in all respects only as illustrative and not restrictive.

What is claimed is:

1. A cyclic heat therapy device comprising:
one or more thermoelectric cooling elements having a top and bottom portion;
one or more heat sinks in thermal connection with top portion of the thermoelectric cooling elements;
one or more control circuits in connection to said thermoelectric cooling elements;
one or more thermally conductive external interface surfaces in thermal connection with bottom portion of thermoelectric cooling elements; and
a means for securing the thermally conductive external interface surfaces to the body of a human or animal

2. The cyclic heat therapy device of claim 1 where in the control circuits contain circuitry and electronic components to allow for the cycling of the thermoelectric cooling elements between heating and cooling states.

3. The cyclic heat therapy device of claim 1 where in the control circuits contain user interface switches, buttons, lights, or displays to allow the user to select and visualize heating, cooling, and cycling modes of operation.

4. The cyclic heat therapy device of claim 1 where in the control circuits contain circuitry and electronic components to set and control the time duration of heating and cooling states.

5. The cyclic heat therapy device of claim 1 where in the control circuits contain circuitry and electronic components to measure, set, and control the temperature of the thermally conductive external interface surfaces.

6. The cyclic heat therapy device of claim 1 where in the control circuits contain circuitry and electronic components to measure, set, and control the temperature of the heat sinks.

7. The cyclic heat therapy device of claim 1 where in the means for securing the thermally conductive external interface surfaces to the body are composed of one or more adhesive interface surfaces adjacent or attached to the thermally conductive external interface surfaces.

8. The cyclic heat therapy device of claim 1 where in the means for securing the thermally conductive external interface surfaces to the body are composed of one or more flexible wraps.

9. The cyclic heat therapy device of claim 1 where the control circuits are contained between the heat sinks and the thermally conductive external interface surfaces.

10. The cyclic heat therapy device of claim 1 where the control circuits are located in a region outside of the area between the heat sinks and the thermally conductive external interface surfaces.

11. A cyclic heat therapy device comprising:
one or more thermoelectric cooling elements having a top and bottom portion;
one or more heat sinks in thermal connection with top portion of the thermoelectric cooling elements;
one or more control circuits in connection to said thermoelectric cooling elements comprising circuitry and electronic components to allow for the automated cycling of the thermoelectric cooler between heating and cooling states;
one or more thermally conductive external interface surfaces in thermal connection with bottom portion of thermoelectric cooling elements; and
a means for securing the thermally conductive external interface surfaces to the body of a human or animal.

12. The cyclic heat therapy device of claim 11 where in the control circuits contain user interface switches, buttons, lights, or displays to allow the user to select and visualize heating, cooling, and cycling modes of operation.

13. The cyclic heat therapy device of claim 11 where in the control circuits contain circuitry and electronic components to set and control the time duration of heating and cooling states.

14. The cyclic heat therapy device of claim 11 where in the control circuits contain circuitry and electronic components to measure, set, and control the temperature of the thermally conductive external interface surfaces.

15. The cyclic heat therapy device of claim 11 where in the control circuits contain circuitry and electronic components to measure, set, and control the temperature of the heat sinks.

16. The cyclic heat therapy device of claim 11 where the means for securing the thermally conductive external interface surfaces to the body are composed of one or more adhesive interface surfaces adjacent or attached to the thermally conductive external interface surfaces.

17. The cyclic heat therapy device of claim 11 where the means for securing the thermally conductive external interface surfaces to the body are composed of one or more flexible wraps.

18. A cyclic heat therapy device comprising:
one or more thermoelectric cooling elements having a top and bottom portion;
one or more heat sinks in thermal connection with top portion of the thermoelectric cooling elements and forming the upper cover of the device;
one or more control circuits in connection to said thermoelectric cooling elements comprising circuitry and electronic components to allow for the automated cycling of the thermoelectric cooler between heating and cooling states;
one or more thermally conductive external interface surfaces in thermal connection with bottom portion of thermoelectric cooling elements and forming the bottom portion of the device; and
a means for securing the thermally conductive external interface surfaces to the body of a human or animal

19. The cyclic heat therapy device of claim 18 where the means for securing the thermally conductive external interface surfaces to the body are composed of one or more adhesive interface surfaces adjacent or attached to the thermally conductive external interface surfaces.

20. The cyclic heat therapy device of claim 18 where the means for securing the thermally conductive external interface surfaces to the body are composed of one or more flexible wraps.
